(19) United States
(12) Patent Application Publication
(10) Pub. No.: US 2025/0295292 A1
(43) Pub. Date: Sep. 25, 2025

(54) MEDICAL DEVICES, SYSTEMS, AND METHODS INCLUDING HAPTIC FEEDBACK

(71) Applicant: Boston Scientific Scimed, Inc., Maple Grove, MN (US)

(72) Inventor: Guilherme MILITZ, Cashel (IE)

(73) Assignee: Boston Scientific Scimed, Inc., Maple Grove, MN (US)

(21) Appl. No.: 19/083,967

(22) Filed: Mar. 19, 2025

Related U.S. Application Data
(60) Provisional application No. 63/567,628, filed on Mar. 20, 2024.

(51) Int. Cl. A61B 1/00; A61B 1/06; A61B 90/00

(52) U.S. Cl. CPC A61B 1/00066; A61B 1/00006; A61B 1/00052; A61B 1/00055; A61B 1/00097; A61B 1/0676; A61B 90/06; A61B 2090/064; A61B 2560/0462; A61B 2560/0487; A61B 2562/0219; A61B 2562/0247; A61B 2562/0271

(57) ABSTRACT

Medical devices and related methods are described. A medical device may include a handle including a haptic feedback module, the haptic feedback module configured to generate a first haptic feedback pattern upon satisfaction of at least one condition, and a shaft extending distally from the handle, a distal end of the shaft including at least one measurement sensor. The at least one measurement sensor may be configured to measure a first parameter associated with a target site of a subject.

### CROSS-REFERENCE TO RELATED APPLICATION(S)

This application claims the benefit of priority to U.S. Provisional Application No. 63/567,628, filed on Mar. 20, 2024, which is incorporated by reference herein in its entirety.

### TECHNICAL FIELD

Various aspects of the present disclosure relate generally to systems, devices, and methods used in medical procedures. More specifically, this disclosure relates to medical devices capable of haptic feedback generation, and related systems and methods.

### BACKGROUND

Medical professionals using ureteroscopes, endoscopes, and other medical devices often need to divert their attention from a patient during a medical procedure to monitor sensor data. For example, a medical professional may turn their attention from a viewing screen to check intraluminal pressure in a body orifice (e.g., a kidney), which may cause procedure complications and increase procedure time. The absence of non-visual sensor feedback can decrease procedural accuracy, delay user reaction time, increase the chance of procedural complications, and increase procedure complexity.

### SUMMARY

Examples of the present disclosure relate to, among other things, systems, devices, and methods for generating haptic feedback during a medical procedure. Each of the examples disclosed herein may include one or more of the features described in connection with the disclosed examples.

For example, a medical device may include a handle including a haptic feedback module, the haptic feedback module configured to generate a first haptic feedback pattern upon satisfaction of at least one condition, and a shaft extending distally from the handle, a distal end of the shaft including at least one measurement sensor. The at least one measurement sensor may be configured to measure a first parameter associated with a target site of a subject.

Any of the systems, devices, and methods disclosed herein may include any of the following features. The medical device may include a control unit configured to determine, based on the measured parameter associated with the target site, that the at least one condition has been satisfied. The control unit may be configured to determine, based on a second measured parameter, that a second condition has been satisfied. The haptic feedback module is configured to generate a second haptic feedback pattern upon satisfaction of the second condition. The first condition and the second condition may be associated with the first parameter. The second condition may have a greater value or magnitude than the first condition. The second haptic feedback pattern may have a greater vibrational amplitude and/or frequency than the first haptic feedback pattern. The distal end may include an illuminator configured to flash upon satisfaction of the at least one condition. The at least one measurement sensor may include a thermometer. The at least one measurement sensor may include a pressure sensor. The at least one measurement sensor may further include at least one of an accelerometer, a gyroscope, a carbon dioxide sensor, an oxygen sensor, a pH sensor, an impedance sensor, a chemical sensor, a force sensor, or a radar sensor. The at least one condition may be based on at least the first measured parameter and the second measured parameter. The second haptic feedback pattern may have a different vibrational pattern than the first haptic feedback pattern. The first haptic feedback pattern may be based on a user identity or credential. The haptic feedback module may include a vibration motor.

This disclosure also includes a method. The method may include inserting a medical device into a subject. The medical device may include one or more sensors coupled to a distal end of a shaft of the medical device, the one or more sensors each configured to measure one or more parameters. The method may include positioning a distal portion of the shaft at a target site, measuring the one or more parameters with the one or more sensors of the medical device, determining, based on the one or more measured parameters, that a first condition has been satisfied, and upon satisfaction of at least the first condition, generating haptic feedback.

Any of the systems, devices, and methods disclosed herein may include any of the following features. The method may include determining, based on the one or more measured parameters, that a second condition has been satisfied. The haptic feedback may be a first haptic feedback pattern, and the method may further include upon satisfaction of at least the second condition, generating a second haptic feedback pattern.

In other aspects, a method may include inserting a medical device into a subject. The medical device may include one or more sensors coupled to the medical device, the one or more sensors may each be configured to measure one or more parameters. The method may further include positioning a distal portion of a shaft of the medical device at a target site, measuring the one or more parameters with the one or more sensors of the medical device, comparing at least one of the one or more measured parameters to two or more thresholds, each of the two or more thresholds associated with a different numerical value range than each other of the two or more thresholds, a first threshold having a lowest associated numerical value range of the two or more thresholds and a second threshold having a greatest numerical value range of the two or more ordered thresholds, determining a highest threshold satisfied by the measured one or more parameters, and upon the determining, generating a haptic feedback pattern based on the determined highest threshold.

Any of the systems, devices, and methods disclosed herein may include any of the following features. The highest threshold may include a first highest ordered threshold, further including, subsequent to the first determining, determining a second highest ordered threshold satisfied by the measured one or more parameters, the second highest ordered threshold different than the first highest ordered threshold.

### BRIEF DESCRIPTION OF THE DRAWINGS

The accompanying drawings, which are incorporated in and constitute a part of this specification, illustrate various exemplary embodiments and together with the description, serve to explain the principles of the disclosure.

FIG. 1 depicts an exemplary medical device, according to aspects of this disclosure.

FIG. 2 depicts an exemplary distal end portion of the medical device of FIG. 1, according to aspects of this disclosure.

FIG. 3 depicts a graph of sensor data over time, according to aspects of this disclosure.

FIG. 4 provides a flowchart depicting an exemplary method for haptic feedback generation during a medical procedure, according to aspects of this disclosure.

### DETAILED DESCRIPTION

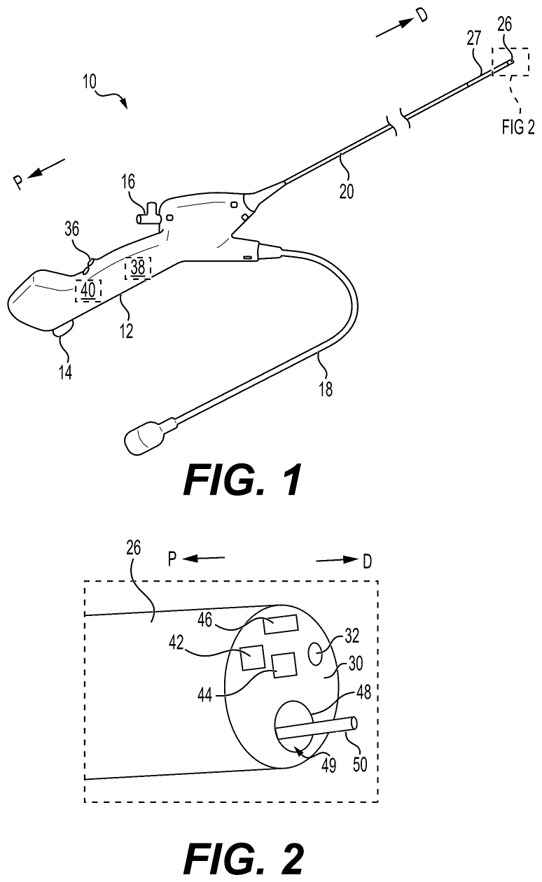

It may be understood that both the foregoing general description and the following detailed description are exemplary and explanatory only and are not restrictive of the invention, as claimed. As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. Proximal and distal directions are labeled with arrows marked “P” and “D”, respectively, in FIGS. 1 and 2. The terms “about,” “approximately,” and like terms (e.g., “substantially”) include values+/−10% of a stated value.

Aspects of this disclosure provide configurations of a handle and a distal end portion of a medical device, including one or more sensors and other components configured to generate haptic feedback based on sensor data, e.g., to facilitate treatment of a subject. A medical device, such as an ureteroscope, may be inserted into a body lumen of a subject (e.g., into a kidney) in order to perform a medical procedure (e.g., a laser lithotripsy procedure). A distal end portion of the medical device may include one or more electronic components, such as light sources (e.g., light emitting diodes (LEDs)), imaging devices (e.g., cameras, other components having imagers, and/or other optical elements such as lenses), and/or other electronic components (e.g., circuit board(s), capacitors, diodes, resistors, etc.). Electronic components of the devices herein may include electrical connections, and may also include various elements mounted, for example, or otherwise connected to, the electrical connections. Aspects of the devices of this disclosure may enable haptic feedback to be provided to a user during a medical procedure.

In some examples herein, including those discussed below, a distal end portion of a medical device (e.g., a distal end portion of a shaft of the medical device) may comprise one or more sensors configured to detect one or more conditions. The one or more sensors may communicate measured parameter data to a control unit of the medical device or connected to the medical device. The control unit may control a haptic feedback device to provide haptic feedback to a user.

FIGS. 1 and 2 depict aspects of an exemplary medical device 10 according to the present disclosure. Although the discussion herein may refer to a ureteroscope as a medical device with the features disclosed herein, it will be appreciated that, unless otherwise specified, the features herein may be used on other suitable medical devices such as, e.g., endoscopes, duodenoscopes, gastroscopes, endoscopic ultrasonography (“EUS”) scopes, colonoscopes, bronchoscopes, laparoscopes, cystoscopes, aspiration scopes, catheters, and any other suitable medical devices. FIG. 1 depicts medical device 10, including a handle 12, and a shaft 20 extending distally from the handle 12. A portion of shaft 20 is omitted in FIG. 1 purely for illustration purposes, and shaft 20 may be any suitable length. FIG. 2 depicts a magnified view of a distal end portion 26 of shaft 20. Handle 12 may be configured for gripping and use by an operator (e.g., a medical professional).

At least a portion of shaft 20 may be flexible, for example, to facilitate navigation through a subject's anatomy, and may define one or more lumens. In some aspects, shaft 20 may include a lumen configured to serve as a working channel for delivery of various instruments and/or material(s) (e.g., fluid) through an opening 48 at distal end 26 of the shaft 20. Shaft 20 may be configured for at least partial insertion into a body (e.g., a body lumen) of a subject. Shaft 20 may include an articulation section 27 at and/or proximate to distal end portion 26, and articulation section 27 may be configured to bend via actuation of one or more actuators, such as via actuation of actuator 14 of handle 12. Articulation section 27 may facilitate maneuvering shaft 20 through a patient's body.

As discussed in further detail below, the distal end portion 26 of shaft 20 may include one or more electronic components operably connected to handle 12, e.g., to allow a user control of the electronic component(s) via one or more actuators of the handle 12. Furthermore, one or more articulation members (e.g. wires) may extend from handle 12 through shaft 20 to articulate shaft 20 in one or more directions. Handle 12 may include an actuator 14 (e.g., lever, switch, button, etc.) coupled to the articulation member(s) to direct shaft 20 in the one or more directions.

A port 16 of handle 12 may provide access to a lumen (e.g., working channel) of shaft 20 of medical device 10. An operator may insert an instrument or deliver a material to the lumen via port 16. As mentioned above, the lumen may extend longitudinally through shaft 20 and terminate at a distal opening 48 of a distal end 30 of shaft 20. Handle 12 may include one or more other actuators, such as actuator 36, operably coupled to an imaging device 32 of shaft 20 to capture still and/or video images. An umbilicus 18 coupled to handle 12 may provide power, signals, etc. to and/or from handle 12. For example, umbilicus 18 may connect medical device 10 to one or more user interfaces, monitors, control units, displays, etc., via handle 12. In some examples, imaging device 32 and/or any other electrical components at distal end portion 26 may be actuated via a remote system, such as a control unit, monitor, or other user interface, connected to medical device 10 through umbilicus 18 or wirelessly connected to medical device 10 (e.g. via one or more transmitters and/or receivers positioned within handle 12).

A control unit 38 may receive data measured by one or more sensors of medical device 10, such as one or more sensors on distal end 30. Control unit 38 may be positioned entirely within handle 12, may be positioned in other portions of medical device 10, or may be a remote system in communication with medical device 10 (via umbilicus 18, wirelessly, and/or via any other means known in the art). Control unit 38 may be configured to determine whether received sensor data satisfies one or more thresholds (e.g. a threshold pressure level, etc.). Control unit 38 may be configured to transmit signaling to haptic feedback module 40. The signaling transmitted to haptic feedback module 40 may correspond to specific vibration patterns and intensities to be generated by haptic feedback module 40.

Control unit 38 may be any suitable electronic device capable of associating a specific input with a specific output. In some embodiments, control unit 38 may be a logic gate. In other embodiments, control unit 38 may be a microprocessor or system on a chip (SoC) configured to execute one or more lines of code.

Control unit 38 may utilize circuitry (e.g., one or more circuits) in order to implement standards, protocols, methodologies, and/or technologies disclosed herein, operably couple two or more components, generate information, process information, analyze information, generate signals, encode/decode signals, convert signals, transmit and/or receive signals, control other devices, etc. Circuitry of any type can be used, and portions of circuitry may include one or more wireless transmitters to connect wirelessly to one or more remote systems.

In an embodiment, circuitry includes, among other things, one or more computing devices such as a processor (e.g., a microprocessor), a general purpose computation on graphics processing unit (GPGPU), a digital signal processor (DSP), an application-specific integrated circuit (ASIC), a field-programmable gate array (FPGA), a SoC, or the like, or any combinations thereof, and can include discrete digital or analog circuit elements or electronics, or combinations thereof. In an embodiment, circuitry includes hardware circuit implementations (e.g., implementations in analog circuitry, implementations in digital circuitry, and the like, and combinations thereof).

In an embodiment, circuitry includes combinations of circuits and computer program products having software or firmware instructions stored on one or more computer readable non-transient memories that work together to perform one or more protocols, instructions, methodologies or technologies described herein. In an embodiment, circuitry includes circuits, such as, for example, microprocessors or portions of microprocessor, that require software, firmware, and the like for operation. In an embodiment, circuitry includes one or more processors or portions thereof and accompanying software, firmware, hardware, and the like. It should be understood that while control unit 38 is depicted as a component of medical device 10, one of ordinary skill in the art will appreciate that control unit 38 may be located in an external device, such as a computer or distributed computing array (e.g., a cloud server) that is communicatively coupled to medical device 10 via umbilicus 18 and/or via wireless communication with medical device 10.

Haptic feedback module 40 may be a vibration motor, a micro vibration module, a vibrotactile device, a force feedback device, a haptic actuator, a vibration feedback device, a kinesthetic feedback device, and/or the like for transmitting haptic feedback to a user. Haptic feedback module 40 may generate various haptic feedback patterns, tactile patterns, vibrotactile sequences, tactile rhythms, tactile cues, haptic codes, vibrational profiles (e.g., vibration patterns associated with a specific user), and the like at various levels of vibrational intensity, amplitude, magnitude, frequency, and pattern. While haptic feedback module 40 is depicted as a component of handle 12, this is only exemplary. Haptic feedback module 40 may be positioned entirely within handle 12, may be positioned in any other portion of medical device 10, and/or may be in a remote system (e.g. wristwatch, chest strap, glove, etc.). In some embodiments, haptic feedback module 40 may comprise an external device that may be worn or otherwise fixedly attached to a user (e.g., a wristband, glove, eyewear, or chest strap). In this external embodiment, signaling from control unit 38 may cause haptic feedback module 40 to vibrate. This embodiment may be utilized to retrofit existing medical devices with haptic feedback functionality. In some examples, feedback module 40 may be incorporated into control unit 38, for example contained on a circuit board comprising control unit 38 or otherwise a part of control unit 38. Haptic feedback module 40 may be adjustable, such that a user may select a type and/or degree of feedback (e.g. a vibration pattern, a vibration intensity, etc.). In some examples, haptic feedback module 40 may provide other types of feedback, such as audio or visual feedback, in combination with haptic feedback or may only provide audio or visual feedback without haptic feedback.

In some embodiments, haptic feedback patterns (including any aforementioned characteristics of haptic feedback) may be based on a user identity or credential. For example, a first user may prefer their haptic feedback at a first intensity I, and a second user may prefer their haptic feedback at a greater intensity 21.

As shown in FIG. 2, distal end portion 26 may include a distal end 30 that defines an opening 48 of a lumen (working channel) 49. Instruments and/or other material(s) may be passed through port 16, through the lumen, and extend out of opening 48. An instrument extending distally of opening 48 may be used to perform a medical procedure on the subject.

A tool 50 may be inserted into a working channel or lumen 49 of medical device 10, and tool 50 may exit out of the distal end of lumen 49 at opening 48. Tool 50 may include, for example, a brush, such as a wire brush, a guidewire, cutting or grasping forceps, a biopsy device, a snare loop, an injection needle, a cutting blade, an electrosurgical knife, scissors, a retractable basket, a retrieval device, an ablation and/or electrophysiology catheter, a stent placement device, a surgical stapling device, a balloon catheter, a laser-emitting device, and/or any other suitable therapeutic or diagnostic accessory device.

Distal end 30 of distal end portion 26 may include an imaging device 32 (e.g. a camera, such as a CMOS or CCD camera). Imaging device 32 may be configured to take video and/or still images. Imaging device 32 may provide a signal to a monitor or display (e.g., connected via umbilicus 18), so that an operator may view an image provided by imaging device 32 while navigating medical device 10 through a body lumen of a subject. An operator may take photos and/or videos using imaging device 32 via an actuator 36, or imaging device 32 may be actuated by a remote system or other actuator of handle 12. One of skill in the art will appreciate that imaging device 32 may include various lenses and/or sensors. Imaging device 32 may output data to control unit 38.

Distal end 30 of distal end portion 26 may include one or more illuminators 46, which may include light emitting diodes (LEDs) or the like. In some examples, one or more windows (not shown) may be in front of the one or more illuminators 46. In some examples, flashing of illuminator at a specific rate may indicate when sensor data of medical device 10 exceeds a threshold value, in coordination with haptic feedback from haptic feedback module 40 or separate from haptic feedback module 40.

Distal end 30 of distal end portion 26 may include various sensors. The various sensors may measure various conditions present at the distal end 30 of the medical device 10. The various sensors may be communicatively coupled to control unit 38 and/or the various other sensors. The various sensors may transmit measured parameters and/or patterns to control unit 38. For example, a temperature sensor 42 is shown on the distal end 30. Temperature sensor 42 may measure temperatures at various positions in a body orifice as a user orients and/or navigates distal end portion 26 through patient anatomy. Temperature sensor 42 may be an infrared thermometer or any other suitable sensor for recording temperatures inside of a subject.

Also shown on distal end 30 is a pressure sensor 44. Pressure sensor 44 may be configured to record various pressure readings at various positions in a body orifice as a user orients and/or navigates distal end portion 26 through patient anatomy. Pressure sensor 44 may measure, for example, an intraluminal pressure in a kidney of a subject.

While temperature sensor 42 and pressure sensor 44 are shown in FIG. 2, one of ordinary skill in the art will appreciate that various other sensors may be utilized with the exemplary embodiments. For example, the various sensors may include, an accelerometer and/or gyroscope for measuring orientation of distal end 30, a carbon dioxide sensor, an oxygen sensor, a pH sensor to measure acidity and/or alkalinity, an impedance sensor (e.g. for measuring changes in tissue composition), chemical sensors configured to detect the presence and concentration of various chemicals, force sensors configured to measure the force exerted by medical device 10 or another medical instrument on patient anatomy, LIDAR and/or radar sensors to measure distances from distal end 30 to target anatomy, and the like. Any suitable combination of sensors may be utilized at distal end 30. Any of the sensors of medical device 10 may be actuated to collect sensor data, or may automatically collect sensor data.

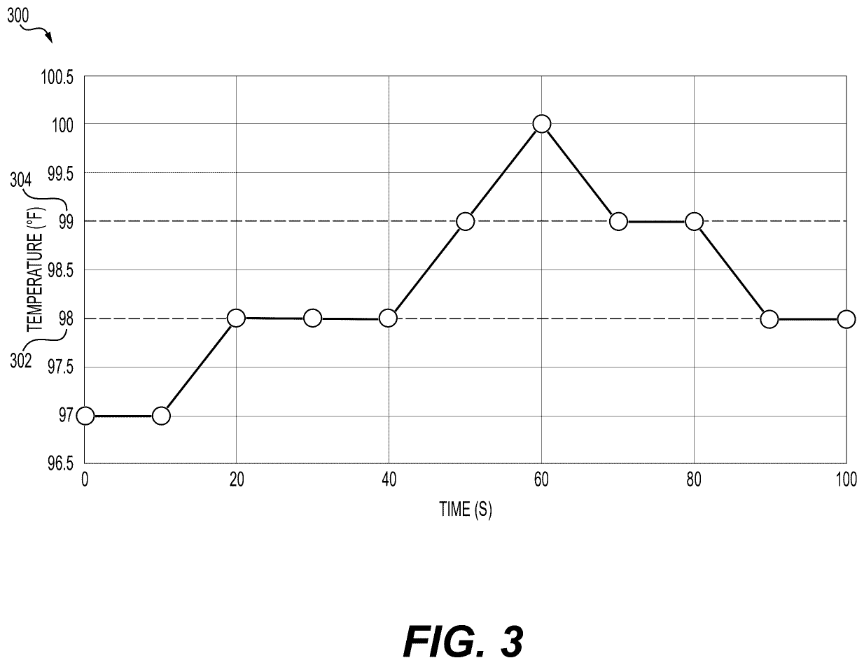

FIG. 3 depicts a graph 300 of sensor data over time according to aspects of this disclosure. Graph 300 is only exemplary, one of ordinary skill in the art will appreciate that other metrics, parameters, and patterns may be detected by the one or more sensors for processing by the control unit 38.

Graph 300 illustrates exemplary temperature measured over time (e.g., 100 seconds) during a medical procedure, though the depicted length of time and temperature range are provided only as an example. At time (t) t=0, a medical professional may have distal end 30 located at a first position inside the subject. At t=0, a sensor (e.g., temperature sensor 42) located on distal end 30 may measure a temperature of 97 degrees Fahrenheit.

Shown on graph 300 is a first threshold 302, which corresponds to a temperature of 98 degrees Fahrenheit. One of ordinary skill in the art will appreciate that the depicted threshold values in FIG. 3 are exemplary; various quantities and metrics may be utilized by any of the exemplary embodiments with one or more of the various sensors on distal end 30. For example, control unit 38 may be monitoring data output by multiple sensors (e.g., temperature sensor 42 and pressure sensor 44) to determine whether to trigger haptic feedback module 40. It should be understood that the various sensors may have various polling rates and that control unit 38 processes the output data as soon as it receives the data from the various sensors. While the polling rate of temperature sensor 42 is depicted as every ten seconds (shown by the circles on the line of graph 300), this is only exemplary. In some embodiments, each measured change in a measured parameter and/or pattern may correspond to a change in haptic pattern and/or intensity, thereby forming a near-continuous haptic response curve (e.g., arbitrarily small threshold values). In some embodiments, threshold values may be adjustable.

Various thresholds may be associated with various vibrational patterns and intensities. For example, at t=20 seconds through t=40 seconds, the measured temperature is equal to first threshold 302 of 98 degrees. First threshold 302 may be associated with a first haptic feedback pattern and intensity (H₁). H₁ may be maintained in handle 12 via haptic feedback module from t=20 to t=50. At t=50, temperature sensor 42 measures a temperature of 99 degrees. This temperature may correspond with a second threshold 304. Second threshold 304 may be associated with a second haptic feedback pattern and intensity (H₂). H₂ may be maintained in handle 12 via haptic feedback module from t=50 to t=90. In general, higher thresholds may correspond with stronger, and/or greater frequency, and/or greater intensity haptic feedback than lower thresholds, though this is only exemplary. It should be understood that maintaining a haptic feedback pattern and intensity outside of its corresponding threshold is possible, based on the polling rate of the sensor associated with a given threshold. As soon as control unit 38 is aware of a change in a parameter corresponding to a different threshold than a currently occupied threshold, control unit 38 updates the haptic feedback at haptic feedback module 40. Thresholds may be defined as at or above a specific threshold value, at or below a specific threshold value, or may only trigger haptic feedback when the sensor data is at the specified threshold (and not above or below the specified threshold).

At t=90 to t=100, temperature sensor 42 measures a temperature of 98 degrees, corresponding to first threshold 302 and H₁. While thresholds shown in FIG. 3 are inclusive of associated measured parameter values (e.g., first threshold 302 is greater than or equal to 98 degrees Fahrenheit), it is further contemplated that one or more thresholds may only apply to values greater than the associated measured parameter value (e.g., first threshold 302 is greater than 98 degrees Fahrenheit), or only apply to values less than the associated threshold value.

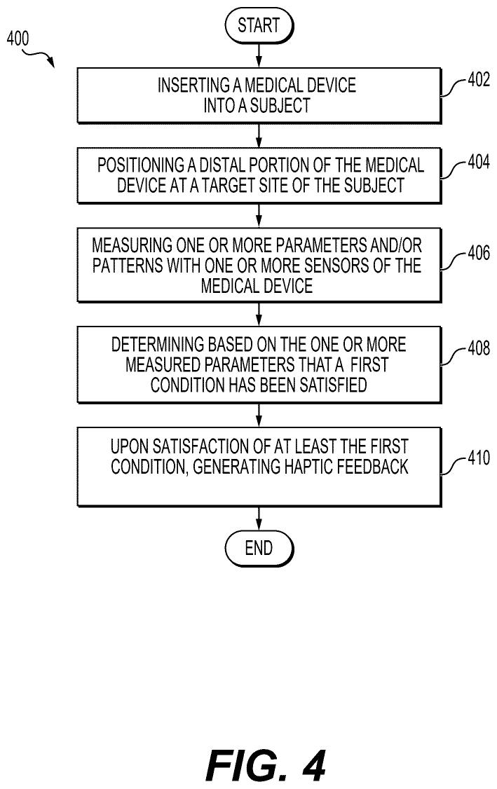

FIG. 4 provides a flowchart 400 depicting an exemplary method for haptic feedback generation during a medical procedure, according to aspects of the disclosure.

In 402, a user (e.g., a medical professional) may insert a medical device such as medical device 10 into a subject (e.g., a kidney). This insertion may be part of a medical procedure such as a ureteroscopy, and may be through a natural orifice or an incision.

In 404, the user positions a distal end portion (e.g., distal end portion 26) of the medical device at a target site of the subject, such as a kidney. The user may navigate the distal portion through the anatomical curves and/or through one or more body lumens of the subject to the target site.

In 406, one or more sensors coupled to a distal end (e.g., distal end 30) of the distal portion measure one or more parameters and/or patterns at the target site. For example, a temperature sensor (e.g., temperature sensor 42) may measure temperatures at the target site as the user manipulates distal end portion 26 at the target site as part of the medical procedure.

In 408, the medical device determines, based on the one or more measured parameters by the one or more sensors, that a first condition (e.g., a threshold) has been satisfied. For example, a temperature threshold may have been satisfied or exceeded, as shown in FIG. 3.

In 410, upon satisfaction of at least the first condition, the medical device generates haptic feedback. For example, the medical device may generate haptic feedback in a handle portion (e.g., handle 12) of the medical device, and/or may generate haptic feedback in another portion of the medical device or in a remote system (e.g. a wristwatch, glove, chest strap, etc.). The haptic feedback pattern and intensity may be associated with a given condition.

Providing haptic feedback in a medical device allows a user to keep their attention on imaging from the patient and not repeatedly divert their gaze to monitor sensor output data. Systems, devices, and methods of this disclosure may prevent procedural complications, reduce procedure time, and prevent medical professional mistakes during a medical procedure, for example by allowing a user to maintain focus on an imaging monitor and simultaneously become aware of an undesirable condition in the patient, such as a dangerous temperature increase or a dangerous pressure level within the body of the patient.

While principles of this disclosure are described herein with the reference to illustrative examples in a particular context and for particular medical procedures, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

1. A medical device comprising:
a handle including a haptic feedback module, the haptic feedback module configured to generate a first haptic feedback pattern upon satisfaction of at least one condition; and
a shaft extending distally from the handle, a distal end of the shaft including at least one measurement sensor, wherein the at least one measurement sensor is configured to measure a first parameter associated with a target site of a subject.

2. The medical device of claim 1, further comprising:
a control unit configured to determine, based on the measured parameter associated with the target site, that the at least one condition has been satisfied.

3. The medical device of claim 2, wherein the control unit is configured to determine, based on a second measured parameter, that a second condition has been satisfied.

4. The medical device of claim 3, wherein the haptic feedback module is configured to generate a second haptic feedback pattern upon satisfaction of the second condition.

5. The medical device of claim 4, wherein the first condition and the second condition are associated with the first parameter.

6. The medical device of claim 4, wherein the second condition has a greater value or magnitude than the first condition.

7. The medical device of claim 4, wherein the second haptic feedback pattern has a greater vibrational amplitude and/or frequency than the first haptic feedback pattern.

8. The medical device of claim 1, wherein the distal end includes an illuminator configured to flash upon satisfaction of the at least one condition.

9. The medical device of claim 1, wherein the at least one measurement sensor comprises a thermometer.

10. The medical device of any of claim 1, wherein the at least one measurement sensor comprises a pressure sensor.

11. The medical device of claim 1, wherein the at least one measurement sensor further comprises at least one of an accelerometer, a gyroscope, a carbon dioxide sensor, an oxygen sensor, a pH sensor, an impedance sensor, a chemical sensor, a force sensor, or a radar sensor.

12. The medical device of claim 3, wherein the at least one condition is based on at least the first measured parameter and the second measured parameter.

13. The medical device of claim 4, wherein the second haptic feedback pattern has a different vibrational pattern than the first haptic feedback pattern.

14. The medical device of claim 1, wherein the first haptic feedback pattern is based on a user identity or credential.

15. The medical device of claim 1, wherein the haptic feedback module comprises a vibration motor.

16. A method, comprising:
inserting a medical device into a subject, wherein the medical device includes one or more sensors coupled to the medical device, the one or more sensors each configured to measure one or more parameters;
positioning a distal portion of a shaft of the medical device at a target site;
measuring the one or more parameters with the one or more sensors of the medical device;
determining, based on the one or more measured parameters, that a first condition has been satisfied; and
upon satisfaction of at least the first condition, generating haptic feedback.

17. The method of claim 16, further comprising:
determining, based on the one or more measured parameters, that a second condition has been satisfied.

18. The method of claim 17, wherein the haptic feedback is a first haptic feedback pattern, and further comprising:
upon satisfaction of at least the second condition, generating a second haptic feedback pattern.

19. A method, comprising:
inserting a medical device into a subject, wherein the medical device includes one or more sensors coupled to the medical device, the one or more sensors each configured to measure one or more parameters;
positioning a distal portion of a shaft of the medical device at a target site;
measuring the one or more parameters with the one or more sensors of the medical device;
comparing at least one of the one or more measured parameters to two or more thresholds, each of the two or more thresholds associated with a different numerical value range than each other of the two or more thresholds, a first threshold having a lowest associated numerical value range of the two or more thresholds and a second threshold having a greatest numerical value range of the two or more ordered thresholds;
determining a highest threshold satisfied by the measured one or more parameters; and
upon the determining, generating a haptic feedback pattern based on the determined highest threshold.

20. The method of claim 19, wherein the highest threshold comprises a first highest ordered threshold, further comprising:
subsequent to the first determining, determining a second highest ordered threshold satisfied by the measured one or more parameters, the second highest ordered threshold different than the first highest ordered threshold.
